(19) United States
(12) Patent Application Publication
(10) Pub. No.: US 2024/0268695 A1
(43) Pub. Date: Aug. 15, 2024

(54) PROCESSING METHOD, DEVICE, AND APPLICATION OF INTRACRANIAL PRESSURE MONITORING AND DATA

(71) Applicant: ZONGYAN HE, Zhangjiagang (CN)

(72) Inventor: HELENA AI HE, New Brunswick, NJ (US)

(21) Appl. No.: 18/108,188

(22) Filed: Feb. 10, 2023

(51) Int. Cl. A61B 5/03; A61B 5/02

(52) U.S. Cl. CPC A61B 5/031; A61B 5/02; A61B 5/6801

(57) ABSTRACT

The invention belongs to the field of intracranial pressure monitoring, and discloses an information processing method, equipment and application for intracranial pressure monitoring, including: using at least two sensing probes, respectively fixed on the skin surface near the jugular vein; Time interval, send the measured jugular venous pulse signal to the host or smart phone via Bluetooth or other methods; analyze the sent signal, find out the corresponding feature points on the jugular venous pulsation map, and record each pulsation and the feature points on each probe The programming software of the host computer calculates the time difference and the mean value of each beat and the characteristic point of each probe; calculates the mean value of intracranial pressure within each sampling time; obtains the change of intracranial pressure by monitoring the change of jugular venous pressure for 24 hours curves for reference.

### TECHNICAL FIELD

The invention falls in the field of intracranial pressure monitoring and, more specifically, relates to information processing, analysis, application, and storage, as well as the physical equipment for intracranial pressure monitoring.

### BACKGROUND

Intracranial pressure, abbreviated as ICP, is the pressure exerted by the contents of the cranial cavity on the walls of the cavity. The lumbar puncture of a normal adult lying on the side in a relaxed state or the pressure in the lateral ventricle when lying on the back is 6˜13.5 mmHg (8.16˜18.36 cmH2O), for children is 3˜6.75 mmHg (4.08˜9.18 cmH2O). Intracranial pressure in an adult lying supine consistently exceeds 15 mmHg (20.4 cmH2O), that is intracranial hypertension. If the intracranial hypertension cannot be detected and treated in time, it can lead to a decrease in cerebral perfusion pressure, a decrease in cerebral blood flow, cerebral ischemia and hypoxia, coma and brain dysfunction, and even brain herniation, endangering the lives of the wounded and sick. Intracranial hemorrhage, brain contusion, cerebral edema, brain swelling, etc. lead to intracranial hypertension in patients with acute craniocerebral trauma, which is the main cause of death and disability of patients. In clinical neurosurgery, ICP monitoring is the most rapid, objective, and accurate method for diagnosing intracranial hypertension, and it is also an important means for observing changes in patients' conditions, early diagnosis, judging operation time, guiding clinical drug treatment, and judging and improving prognosis.

ICP monitoring currently used in clinical practice is all invasive. According to whether the pressure sensor is directly placed in the cranium, ICP monitoring can be divided into the following two categories: \textcircled{1} implantation method, through the skull drilling or craniotomy, the pressure sensor is directly implanted in the brain; \textcircled{2} catheter method, the catheter is placed into the ventricle, cistern or subarachnoid space, the sensor is extracranial, and it is in contact with the fluid or CSF filled in the catheter to measure the pressure. Different pressure sensors convert intracranial pressure into electrical signals and numbers, which can be amplified to display and record ICP.

With the development of medical and engineering technology, ICP has transitioned from invasive to non-invasive monitoring, single to combined, intermittent to continuous, contact to non-contact, and from close to remotely accomplished sensing.

Typical non-invasive ICP monitoring methods include the following:
Clinical imaging examinations
Optic nerve sheath diameter (ONSD) detection
Retinal venous/arterial pressure (RVP or RAP) detection
Transcranial Doppler (TCD)
Flash visual evoked potentials (FVEP)
Tympanic membrane displacement testing (TMDT)
Bregma manometry anterior fontanel pressure (AFP) technology
Cerebral electrical impedance tomography (EIT)
Near-infrared spectroscopy
Other vibration-based detection technologies to detect the cranial cavity's natural/resonance frequency among other vibration-related parameters
Mathematical modeling

As far as development is concerned, non-invasive ICP monitoring is the general trend. However, there is still no satisfactory 24-hour monitoring method shown to be safe, accurate, convenient to use, and economically viable.

### SUMMARY

This invention provides an information processing method, device, and storage medium for intracranial pressure monitoring. More specifically, the processing method includes the following steps:
Step 1, Using at least two sensing probes, respectively fixed on the skin surface near the jugular vein, and the distance between the centers of each probe is greater than or equal to 2 mm;
Step 2: Sending the jugular pulsation signal measured by each probe to the host or smart phone via bluetooth or other wireless or wired methods at regular intervals;
Step 3. The host or smart phone analyzes the signals sent by each probe, finds out the corresponding feature points on the jugular vein pulsation map, and records the time when the same feature point appears on each probe for each pulsation;
Step 4, The programming software of the host computer calculates each beat and the time difference of the same feature point appearing on each probe, and calculates the mean value of the time difference of the same feature point appearing on each probe within the sampling time;
Step 5: Based on the average value of the time difference between the appearance of the same feature point on each probe, a mathematical formula for calculating the average intracranial pressure within the sampling time is established, and through the calibration process compared with other intracranial pressure measurement methods, the host programming software is used to determine the coefficients in the mathematical formula for calculating the average intracranial pressure.
Step 6, Based on the mathematical formula, the programming software of the host calculates the mean intracranial pressure in each sampling time in real time;
Step 7, By monitoring the change of the jugular venous pressure, the intracranial pressure change curve is obtained for reference.

Furthermore, the sensing method adopted in the information processing method of intracranial pressure monitoring is piezoelectric sensing; the sensing probe adopts PVDF piezoelectric film.

Furthermore, the information processing method includes acquiring a jugular vein pulsation map in a manner of array arrangement of sensing elements (Step 3) and using a venous imaging instrument to assist in the positioning of the sensors.

Furthermore, the sensing probe includes sensors pertaining to piezoelectricity, pressure, Doppler ultrasound, photoelectric volume, and other pulse wave sensor technologies. Said jugular vein involves the internal, external, and other jugular veins.

Furthermore, characteristic features on the pulsation maps include the highest, lowest, and peak points of the V-wave as well as time data indicating when pulsation amplitude reaches such characteristic or otherwise specified value.

Furthermore, the sampling method of the information processing method using intracranial pressure monitoring involves using the piezoelectric sensor to trace the electrical signal generated by jugular pulsation, using the electrical signal strength as the amplitude, and recording the continuous jugular pulsation curve of the amplitude with respect to time; the sampling frequency of the electrical signal should not be lower than 500 Hertz.

Furthermore, calculation of the time difference is done as follows:

Put the jugular pulsation amplitude-time curves recorded by sensor probes A and B together, record the time t_{A} and t_{b} when the same characteristic point appears for each sensor probe in each beat, and calculate the time difference |tₐ−t_{b}|.

Next, continuously sample, record, and calculate the time difference (|tₐ−t_{b}|) for a given time period and use the arithmetic mean from the time difference values between all sensor probes as the average time difference t̅_̅{̅a̅b̅}̅ required for intracranial pressure calculation within the given period.

The intracranial pressure y is a function of t̅_̅{̅a̅b̅}̅, expressed as the following:

$$y = f ( \overline{{t}_{ab}} )$$

This function adopts quadratic form with the following formula:

$$y = {a ( \overline{{t}_{a b}} )}^{2} + b ( \overline{{t}_{ab}} ) + c$$

In this formula, a, b, and c are calibration coefficients calculated by comparing the intracranial pressure state below normal, normal, and above normal with the real intracranial pressure value.

Furthermore, said real intracranial pressure can be measured both invasively and non-invasively, and the non-invasive way includes transcranial Doppler ultrasound TCD.

If the intracranial pressure is relatively high, instead use normal, high, and extremely high intracranial pressure states for calibration. Re-calibration is required before each pressure monitoring session and before a different user starts monitoring.

Another objective of the present invention is to provide a device for implementing said intracranial pressure monitoring. This monitoring device has the following components:

The host is used to receive the jugular pulsation signal detected by the sensor probe in real time through wired or wireless methods. After the signal enters the processor for preliminary noise filtering and amplification, it is transmitted to the cloud data processing and storage center through Wi-Fi for further processing. and intracranial pressure calculation, and the settlement result is sent back to the host through Wi-Fi for display.

Sensing probe, described sensing probe is the form of scarf, bib or tie, is provided with digital-to-analog conversion module, bluetooth module and rechargeable battery, and digital-to-analog conversion converts the electric signal that sensing element obtains into digital signal, bluetooth module Real-time transmission of digital signals to the host;

The cloud data processing and storage center is used for in-depth analysis of the digital signals transmitted by the host computer or smart phone in real time, and calculates the statistical data of the patient's current intracranial pressure and intracranial pressure changes in the past period after removing the interference signal, and then sends it back to the host or smartphone to display.

Further, special software needs to be installed in the smart phone, and the software includes version A and version B, version A is suitable for the installation of Android mobile phones, and version B is suitable for the installation of Apple mobile phones;

The application process of the special software includes:
Establish an intracranial pressure detection interface on the smart phone, set personal information input, intracranial pressure calibration and display options on the interface; set up age, height, weight and other physiological parameter input columns in the personal information input options;
In the intracranial pressure display option, set up two display modes: the current intracranial pressure value and the curve of the intracranial pressure change trend in the past period;
At the same time, these data are also transferred to the hospital for reference when professionals treat and monitor the condition, and can also be used as a basis for emergency treatment.

Further, the sensor probe is pasted on the skin near the jugular vein of the patient with good biocompatibility, and the connection with the host includes two types, namely:

(1) Wired connection, connected to the host through data cables and power cables, and setting the power supply in the host;

(2) Wireless connection, the Bluetooth wireless transmission method is used to transmit signals between the host and the probe. The power supply or battery is set in the probe in the form of a scarf, collar or tie. The distance between the host and the probe cannot exceed the power adaptability of the Bluetooth module. the maximum transmission distance.

Another object of the present invention is to provide an application for implementing the intracranial pressure monitoring device, which is applied to home intracranial pressure monitoring or medical intracranial pressure monitoring;

The specific process of the family intracranial pressure monitoring is:

When wearing the sensing element of the intracranial pressure monitoring device, the host or smart phone receives and sends the monitoring data to the cloud data processing and storage center through Wi-Fi, and can decide whether to send the real-time monitoring data of intracranial pressure to the medical unit or relative's mobile phone;

The specific process of the medical intracranial pressure monitoring is:

Multiple sensing elements are used to collect the jugular pulsation signals of multiple patients at the same time, and then send them to the cloud data processing and storage center or the large-scale host in the medical unit through their respective Bluetooth modules; the host is equipped with a multi-channel receiver to simultaneously process each According to the data of each patient, the intracranial pressure is calculated in real time according to the installed special software, and displayed on the host screen. According to the intracranial pressure data of each patient, a warning of intracranial pressure exceeding the limit is issued.

Another object of the present invention is to provide a computer-readable storage medium, which stores a computer program. When the computer program is executed by a processor, the processor executes the steps of the information processing method for monitoring intracranial pressure.

The technical solution to be protected by the present invention has the following advantages and positive effects:

(1) The detection principle of intracranial pressure provided by the present invention is clear and reasonable.

Intracranial pressure is very sensitive to changes in venous pressure. If the jugular vein is compressed during lateral pressure, intracranial pressure will immediately increase. Coughing, sneezing, holding your breath, exerting force, etc. also cause significant fluctuations in intracranial pressure. Therefore, as early as 1936, Pallock and Boshes believed that the formation of intracranial pressure was mainly due to the effect of atmospheric pressure on the large extracranial veins. This explanation is still recognized as more reasonable. The intracranial pressure fluctuates with the beating of the heart, ranging from 0.27-0.53 kPa (2-4 mmHg), which is the result of arterial dilation caused by each beat of the heart. As the breathing action changes, the intracranial pressure also fluctuates slowly, with an amplitude of about 0.7-1.33 kPa (5-10 mmHg), which is the result of venous changes caused by intrathoracic pressure acting on the superior vena cava.

Jugular venous blood pressure is directly related to intracranial pressure and atmospheric pressure. In the case of constant atmospheric pressure, the variation characteristics of intracranial pressure can be obtained through the fluctuation of jugular venous blood pressure. Because the jugular venous blood pressure is much lower than the carotid arterial blood pressure, and the operation of the neck is inconvenient, the conventional arterial blood pressure measurement method cannot be used to measure the jugular venous blood pressure. The present invention draws lessons from the principle that the propagation velocity of arterial pulse wave is positively correlated with arterial pressure, and considers that the velocity of venous pulse wave is also positively correlated with venous blood pressure, and the intracranial pressure information can be obtained accordingly. The principle is as follows:

The jugular vein enters the brain through a hole in the skull and is in direct contact with the cerebrospinal fluid. Changes in intracranial pressure directly affect jugular venous pressure, the two are basically balanced or the former is slightly greater than the latter. When the intracranial pressure rises, the intracranial veins are compressed and more venous blood is squeezed out, resulting in an increase in the tension of the jugular veins, and the more tense the jugular veins, the higher the jugular venous pressure. The pulse wave speed is faster. Therefore, as long as the change of jugular vein pulse wave velocity is measured, the change of intracranial pressure can be known. Two or more pulse wave sensing probes are used. Place it on the skin surface near the jugular vein, measure the same pulse signal at different positions of the jugular vein, and then find out the characteristic points of the jugular vein pulse measured by each probe, such as on the jugular venous pulse (JVP), the peak point of a wave or the trough point of x wave, record the time at which the same beat characteristic point measured by each probe appears, and calculate the time difference between them. When the distance between the probes is fixed, the time difference between the appearance of the pulsation feature point is inversely proportional to the pulse wave propagation velocity, that is, it is inversely proportional to the jugular venous blood pressure, or is inversely proportional to the intracranial pressure. In other words, if the time difference of the beat characteristic points measured between the probes is shorter than the time difference under normal intracranial pressure, it means that the intracranial pressure has increased, and the increase is proportional to the shortened value of the time difference.

(2) The method of the present invention can truly realize 24-hour non-invasive dynamic monitoring of intracranial pressure.

So far, there are two types of non-invasive ICP monitoring methods that have been applied clinically:

1) Clinical manifestations and imaging examinations. Imaging monitoring has the advantages of being objective, accurate, and capable of locating and determining qualitatively, but it is expensive, cannot be used for bedside and continuous monitoring, and is inconvenient to use.

2) Transcranial Doppler (TCD). TCD can reflect the dynamic changes of cerebral blood flow and observe the mechanism of cerebral blood flow self-regulation. However, cerebrovascular activity is affected by many factors, and the relationship between ICP and cerebral blood flow velocity will change. The increase in flow velocity during cerebral vasospasm must be differentiated from cerebral congestion, otherwise it will affect the judgment. In addition, like imaging methods, they are bulky and expensive, cannot be worn and continuously monitored, and are inconvenient to use.

(3) The method proposed by the present invention is different from all non-invasive ICP detection methods so far in terms of working principle, technical means and material components, and has the following innovations:

1) The sensitivity is extremely high, and the intracranial pressure change of 0.1 mmHg caused by the user holding his breath in the toilet or even a slight cough can be measured.

2) The detection range of intracranial pressure is relatively wide, the upper limit is 100 mmHg or close to 80% of the mean arterial pressure.

3) The miniature ultra-thin sensor array can be adhered to the patient's skin surface, and the monitoring signal is transmitted to the host computer in real time through Bluetooth, so it does not affect the daily life of the user, and can truly realize 24-hour dynamic monitoring of ICP.

4) The host uses Wi-Fi to transmit the monitoring data to the cloud center for processing and analysis, and the latter sends the monitoring results back to the host for display, so that the patient's family or the hospital can dynamically manage the condition.

5) This technology can be used in ICP monitoring centers of hospitals at all levels to realize 24-hour dynamic monitoring of the condition of multiple patients.

(4) The method of the present invention has higher safety than other intracranial pressure detection techniques

The existing non-invasive intracranial pressure detection methods all have their limitations and different degrees of harm to the subjects. For example, optic nerve sheath diameter (ONSD) detection technology is not suitable for people with eyeball and optic nerve diseases. Retinal venous or arterial pressure (RVP or RAP) detection techniques are not suitable for papilledema or intraocular pressure higher than venous pressure. Flash visual evoked potentials (FVEP) technology is easily affected by factors such as age, brain metabolic factors, systemic diseases and metabolic disorders; it is not suitable for patients with severe visual impairment, fundus hemorrhage, coma or brain death. The tympanic membrane displacement (TMD) technique overexposure subjects to sound stimuli can cause temporary threshold changes and discomfort. Vibration frequency detection technology needs to excite the skull to vibrate, which can cause discomfort to the patient.

The present invention passively accepts piezoelectric signals generated by jugular venous pulse, does not send electromagnetic waves, ultrasound or other energy, has no side effects, and does not cause any discomfort to patients

(5) The auxiliary evidence of inventiveness of the present invention is also reflected in the following aspects:

1) It is not necessary to measure a complete and ideal pulse wave waveform, but only to find out the characteristic points on the waveform, so the conditions for the probe to contact the skin (skin condition, contact pressure and area, etc.) are not high, and the operation and It is more convenient to use.

2) The present invention does not involve complex and difficult technical means, nor does it involve biochemical drugs, reagents and other uncertain factors that have potential effects on the human body.

3) The present invention can use the existing mature sensing technology, such as piezoelectricity, photoelectricity or ultrasound for signal acquisition, with high reliability.

4) Since the jugular venous pulsation is significantly different from the carotid pulsation, the interference of the latter can be easily ruled out by its waveform characteristics. Coupled with the auxiliary positioning of the vein imaging device, the success rate of effective signal sampling can be further improved.

5) The device of the present invention has simple structure and small volume. The components used are conventional and mature electronic components, no special manufacturing is required, the reliability is strong, and the yield rate is high.

(6) The expected income and commercial value after the transformation of the technical solution of the present invention are:

The method of the invention is applicable to all people whose intracranial pressure exceeds the standard and healthy people, can truly realize non-invasive monitoring of intracranial pressure for 24 hours, and has a wide range of users. Because the equipment involved is relatively simple, the cost is low, the cost performance is high, the market prospect is good, and it has great commercial value.

(7) The technical solution of the present invention solves the technical problems that people have been eager to solve but have not been successful:

The cloud data processing and storage center provided by the invention can store the data of multiple users, which is convenient for large-scale medical institutions to call at any time, and facilitates the synchronous monitoring and management of intracranial pressure of multiple patients. In this case, the host can be directly connected to the computer network, enter the hospital network system, and complete the processing, classification, mapping and storage of massive patient data.

(8) The technical solution of the present invention overcomes the following technical prejudices:

The present invention overcomes part of the technical bias in developing ICP detection equipment. These biases include: intracranial pressure can only be measured by changes directly related to intracranial tissue, such as changes in cerebral hemodynamics, changes in optic nerve sheath diameter, displacement of the tympanic membrane, changes in skull vibration parameters, etc. On the other hand, technical bias comes from the recognition of the significance of the jugular pulsation. Compared with the carotid artery, the average person pays little attention to the clinical value of the jugular vein measurement. Medical workers usually use the observation of jugular vein distension to qualitatively infer some heart problems. No one has ever associated or tested the jugular vein condition with intracranial pressure. A third bias of the average technician is that measuring either carotid or venous pressure is difficult because it is not possible to place a sphygmomanometer cuff around the neck and inflate the cuff like radial or brachial blood pressure does It is impossible to measure the jugular venous pressure by blocking the blood flow.

### BRIEF DESCRIPTION OF DRAWINGS

FIG. 1 is a flowchart of an information processing method for monitoring intracranial pressure provided by an embodiment of the present invention;

FIG. 2 is a layout diagram of a sensor array provided by an embodiment of the present invention;

FIG. 3 is a schematic diagram of a vein provided by an embodiment of the present invention;

FIG. 4 is a schematic diagram of the jugular venous pulse JVP;

FIG. 5 is a continuous JVP curve diagram provided by the embodiment of the present invention;

FIG. 6 is the amplitude-time curve diagram of the internal jugular vein pulse of volunteer A provided by the embodiment of the present invention;

FIG. 7 is a time-domain diagram (ICP-T) of intracranial pressure change of volunteer B provided by the embodiment of the present invention.

### DETAILED DESCRIPTION

In order to make the object, technical solution and advantages of the present invention clearer, the present invention will be further described in detail below in conjunction with the examples. It should be understood that the specific embodiments described here are only used to explain the present invention, not to limit the present invention.

In order to make those skilled in the art fully understand how to implement the present invention, this part is an explanatory embodiment for explaining the technical solution of the claims.

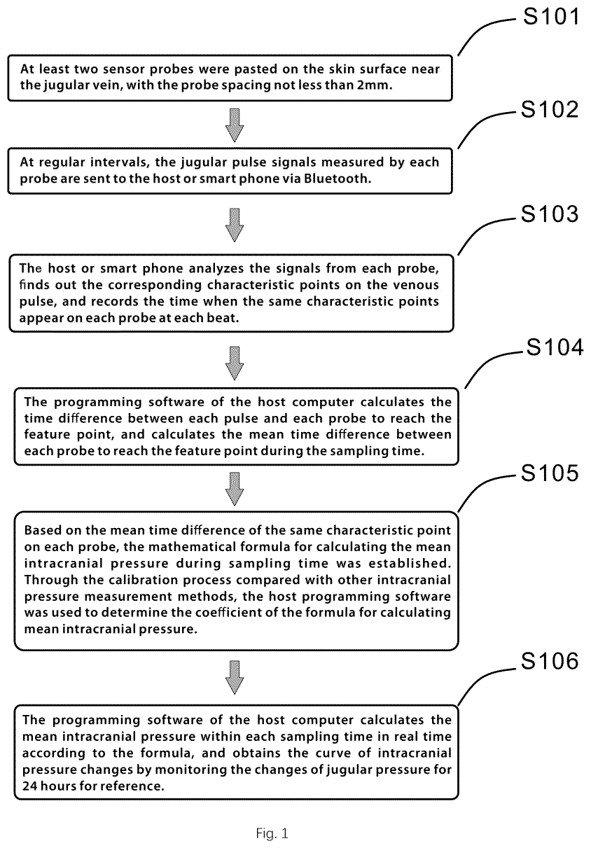

As shown in FIG. 1, the information processing method for monitoring intracranial pressure provided by the embodiment of the present invention includes:
S101, using at least two sensing probes, which are respectively fixed on the skin surface near the jugular vein, and the distance between the centers of the probes is greater than or equal to 2 mm;
S102, sending the jugular vein pulsation signal measured by each probe to the host or the smart phone through bluetooth at regular time intervals;
S103, the host computer or smart phone analyzes the signals sent by each probe, finds out the corresponding feature points on the jugular vein pulse map, and records the time when the same feature point appears on each probe for each beat;
S104, the programming software of the host computer calculates each beat and the time difference of the same feature point appearing on each probe, and calculates the mean value of the time difference of the same feature point appearing on each probe within the sampling time;
S105, based on the mean value of the time difference when the same feature point appears on each probe, establish a mathematical formula for calculating the mean value of intracranial pressure within the sampling time, and determine the intracranial pressure by using host programming software through the calibration process compared with other intracranial pressure measurement methods Coefficient in the mean calculation formula.
S106, the programming software of the host calculates the mean intracranial pressure in each sampling time in real time according to the formula;
By monitoring the change of jugular venous pressure for 24 hours, the intracranial pressure change curve was obtained for reference.

The information processing method using intracranial pressure monitoring can design a complete data receiving-processing-transmitting system by using the aforementioned intracranial pressure monitoring technology. The main components of the system are located in the host. The principle flow of the system is: the jugular pulsation signal detected by the sensor probe is received in real time through wired or wireless means, and the signal will enter the processor for preliminary noise filtering and amplification (as mentioned above, the signal strength amplification will not affect the intracranial pressure calculation), and then transmitted to the cloud data processing and storage center via Wi-Fi for further processing and calculation of intracranial pressure. The settlement result is then sent back to the host for display via Wi-Fi. All the functions of the host can also be replaced by a smart phone, but the smart phone needs to install special software. The software has a smart phone app application software format, and version A is suitable for installation on mobile phones with an Android system. Version B is suitable for installation on iPhones. The functions of this special software include: establishing an intracranial pressure detection interface on the mobile phone, setting user personal information input, intracranial pressure calibration and display options on the interface; setting age, height, weight in the personal information input options and other physiological parameter input fields. In the intracranial pressure display option, two display modes are set up: the current intracranial pressure value and the curve of the intracranial pressure change trend in the past period. In addition, the software can also transfer these data to the hospital for reference when professionals treat and monitor the condition, and can also be used as a basis for emergency treatment.

The sensing probe provided by the embodiment of the present invention includes sensors related to piezoelectricity, pressure sensing, Doppler ultrasonic sensing, photoelectric volume sensing and other pulse wave sensing technologies. The acquisition of the pulse wave signal is generally achieved by measuring the deformation of the skin surface caused by the pulsation. These deformations will cause the piezoelectric crystal or thin film material (such as PVDF piezoelectric film) close to the skin to output corresponding electrical signals; or cause the reflection of the ultrasound beam produces a Doppler signal. Sensing methods developed based on the former are called piezoelectric sensing or pressure sensing. Sensing developed based on the latter is called Doppler ultrasound sensing. There is also a photoelectric volume sensing method, which is based on the change of blood flow volume caused by pulse. Generally speaking, piezoelectric transmission has high sensitivity, and the volume and mass of the sensor can be small, even in the form of a thin film, which is conducive to sticking on the skin surface without affecting the work and rest of the user. It is an ideal pulse wave monitoring sensor. Doppler ultrasound sensing is very sensitive, but it needs to transmit ultrasound. The ultrasound probe is bulky and not easy to wear, and a coupling agent is needed between it and the skin. The method of Photoplethysmography (PPG) is simple, but the sensitivity is poor. The case of the present invention uses piezoelectric sensing, but the method of the present invention does not exclude other pulse wave sensing methods.

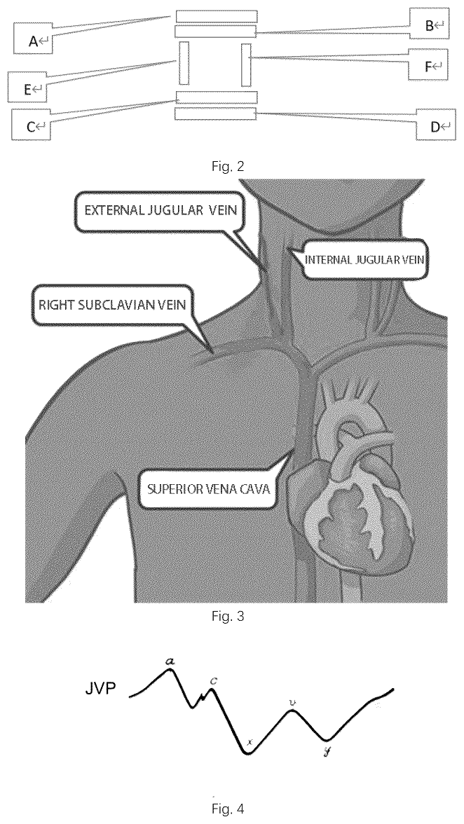

PVDF flexible piezoelectric film, polyvinylidene fluoride piezoelectric film, is a new type of polymer piezoelectric material. The invention adopts PVDF piezoelectric film, which has a large piezoelectric constant (18-30 pC/N), high sensitivity to variable force response, light and flexible film, easy preparation, good impedance coupling with human tissue, and wide temperature range (−30-+80° C.), low mechanical quality factor, small damping, low density, and frequency bandwidth (0.01-100 MHz) can fully meet the frequency characteristics of the pulse signal. The present invention adopts the method of pasting at least two sensing elements along the direction of the jugular vein (as shown in FIG. 2). There is a certain distance H between every two sensors, and H is generally greater than or equal to 2 mm, so that the distances between the positions of different sensors and the heart are obviously different.

The jugular veins provided by the embodiments of the present invention include internal jugular veins, external jugular veins and other jugular veins. The superior vena cava coming out of the right atrium divides into two branches and enters the neck, among which the internal jugular vein enters the cranium (FIG. 3). In the embodiment of the present invention, sensing elements A and B are arranged along the internal jugular vein, and the distance between the centers of the two sensing elements is 10 mm; sensing element B is closer to the heart than sensing element A, and since the pulse wave is sent by the heart, Therefore, B receives the pulse wave signal earlier than A, that is to say, the time T_{A} of a certain feature point of the same beat on A is later than the time TB of the same feature point of this beat on B, and the time difference is T_{A}−T_{B}=t_{AB}>0. Obviously, t_{AB} cannot be less than or equal to zero. However, if the positioning of the vein is not accurate, resulting in sensors A and B not being arranged along the same vein, although sensor B appears to be located closer to the heart than A, due to the different propagation paths of the pulse wave, resulting in changes in the propagation distance, t_{AB} may also occur equal to or even less than zero. In this case, the size of t_{AB} is still related to intracranial pressure, but the calculation formula needs to be adjusted. In addition, if the sampling frequency is low and the signal resolution is insufficient, t_{AB}=0 may also occur.

Since the right internal jugular vein is thicker than the left internal jugular vein, a sensor is installed in the right internal jugular vein of volunteers to measure the pulse wave in the case of the present invention. However, the present invention does not exclude the option of monitoring intracranial pressure at the left internal jugular vein and jugular veins other than the internal jugular vein. Because the jugular venous pulse is far less obvious than the carotid artery, and the internal jugular vein is very close to the carotid aorta, the location of the internal jugular vein is difficult to find. For this reason, the case of the present invention adopts a Venous imaging instrument to assist the positioning of the sensor. The vein imaging device uses the difference in the absorption of near-infrared light by the deoxygenated hemoglobin in the surrounding tissue and the vein. After photoelectric conversion and image processing, the vein is displayed on the screen for medical staff to observe the location of the vein in real time. The vein imaging instrument is not selective to the quality of the patient's veins (elasticity, thickness and depth, etc.), so it is suitable for patients with different diseases and ages. According to the image provided by the imager, the present invention can easily find the position and direction of the internal jugular vein.

The characteristic time point when the pulse wave of the same venous pulsation arrives at each probe provided by the embodiment of the present invention includes the time when the a-wave peak point or the x-wave trough point on the jugular venous pulsation map (JVP) measured by each probe appears. As shown in FIG. 4, JVP can reflect the blood flow of the jugular vein and represent the pressure change during the mechanical activity of the right atrium. JVP is generally obtained by recording the jugular venous pulse with a pressure transducer. It includes three positive waves (a, C, V) and two negative waves (X, Y), and these waves change abnormally during the lesion. The present invention uses the highest point (a wave peak point) or the lowest point (x wave trough point) on the JVP graph as the pulsation feature point, because it is easiest to realize the identification of these points on the graph by programming software. However, the present invention does not exclude the use of other points on the JVP diagram, such as the peak point of the V wave. It is also not excluded to use the time point when the pulsation amplitude reaches a specified value (such as one-half of the maximum amplitude) as the feature point.

The piezoelectric sensor was used to trace the electrical signal value generated by the jugular pulsation, and the electrical signal intensity was used as the amplitude, and the continuous JVP graph curve of the amplitude changing with time was recorded. As shown in FIG. 5, the vertical axis in the figure is the amplitude of the voltage signal intensity, and the higher the amplitude, the greater the skin deformation caused by venous pulsation. The horizontal axis is the time axis. In the figure, Series 2 (red) is the curve measured by the sensor closer to the heart, and Series 1 (blue) is the curve measured by the sensor farther from the heart. It can be clearly seen from the figure that the a-wave peak point of each venous pulsation on the Series 2 curve is ahead of the a-wave peak point on the Series 1 curve. Likewise, the x-trough points on the Series 2 curve all precede the x-trough points on the same beat on the Series 1 curve. The time at which these feature points appear can be easily identified by software, and the time difference between them is also easy to calculate. In order to improve the measurement range and resolution of intracranial pressure, it is necessary to record as many electrical signals as possible from the sensor. But this means that the requirements for data transmission capacity and data storage capacity are very high, so a balance needs to be struck between monitoring performance and hardware cost. Generally speaking, the sampling frequency of electrical signals should not be lower than 500/s.

The elimination of some possible interference factors provided by the embodiments of the present invention, the main interference factors and solutions include:

(1) Correctly identify periodic fluctuations in intracranial pressure caused by each heartbeat through software.

The programming software is used to identify the time difference between two consecutive feature points in each sensor pulse diagram, and the time difference should match the pulse rate or heart rate, which is the reciprocal of the pulse rate or heart rate. If it is found that the difference is too far, it may be a misjudgment of the feature point position, and the value of the feature point time must be canceled or the feature point judgment index must be adjusted.

(2) Correctly identify periodic fluctuations in intracranial pressure caused by breathing.

Because breathing-induced changes in thoracic volume affect the jugular vein, it also affects the venous pulsation curve to produce fluctuations that are roughly consistent with the breathing rhythm. But this kind of fluctuation is small, and generally does not affect the time difference between two consecutive feature points, unless you take a deep breath. In this case, it is necessary to cancel the corresponding feature point time value.

(3) Correctly identify random fluctuations in the pulsation curve caused by singing, speaking loudly, coughing, holding your breath and defecating.

These activities of the subject will produce obvious changes in the beating curve. These changes are random and non-periodic, so they can be ruled out after identification by software.

(4) Identify body position changes

The pulsation curve and intracranial pressure will change greatly after the subject changes from a sitting position to a supine position, especially after the lateral position. The solution is to sample body position sensing devices to identify body position changes, thereby establishing intracranial pressure calculation methods under different body positions.

(5) Interference with sports and similar activities

Due to the high sensitivity of the jugular vein pulse wave sensor, even if the sensing element with a small volume and mass is pasted on the neck, any slight movement of the wearer (such as turning the head, bowing the head, walking or doing exercises) may cause the pulse curve to change. fluctuations, thereby reducing the accuracy of intracranial pressure calculations. For this reason, the present invention adopts the method of timing sampling and calculating intracranial pressure. That is, abandon the practice of continuously sampling the jugular vein pulse 24 hours a day, and change it to sampling at regular intervals. For critically ill patients in the ICU ward, the sampling interval can be set shorter (such as 30 s); for patients recuperating at home, the sampling interval time can be set longer (such as 10 min). In addition, for the dangerous period of increased intracranial pressure (such as defecating), the interval can be appropriately shortened. At the end of the interval and before sampling, the device will issue an early warning, asking the wearer to stop any activity and keep the neck still. Sampling time generally does not exceed 1 minute. After the sampling is finished, a release signal is issued, and the wearer can continue the original activity. In this way, more accurate intracranial pressure values can be obtained at regular intervals. Not only can it effectively eliminate the interference of any slight activity of the human body, improve the accuracy of intracranial pressure measurement, but also reduce the pressure of data processing and storage, reduce device power consumption, and extend battery life.

The time difference required for intracranial pressure provided by the embodiment of the present invention is calculated as:

Put the jugular vein pulse amplitude-time curves recorded by two (or more) sensing probes A and B together, and record the time T_{A} and T_{B} when the same feature point appears in each beat of each two sensing probes, Calculate the time difference between the two T_{A}−T_{B}=t_{AB};

Continuously sample, record and calculate the t_{AB} value within a certain period of time (such as 1 min) (for example, if the heart rate is 60/min, there are 60 values of t_{AB}), and use the arithmetic mean value of these t_{AB} values as the calculation result of the intracranial pressure within this time period. The desired average time difference t_{ABP}.

The calculation method of described intracranial pressure is:

The intracranial pressure Y is a function of t_{ABP}, expressed as:

$$Y = f ( {t}_{ABP} ) \qquad ( 1 )$$

The ƒ(t_{ABP}) mentioned in this case is in the quadratic form, and the formula is:

$$Y = a * {( {t}_{ABP} )}^{2} + b * {t}_{ABP} + c \qquad ( 2 )$$

Among them, the proportional coefficients a, b and c need to be calculated by comparing the three intracranial pressure states (lower than normal, normal and higher than normal) with the real intracranial pressure value of the subject (this process is called calibration). True intracranial pressure can be measured using invasive or other noninvasive methods (such as transcranial Doppler ultrasound TCD). If the patient's intracranial pressure is often at high pressure, normal, high and extremely high intracranial pressure can be selected for calibration. Once the proportionality factor is determined, formula (2) becomes the formula for calculating ICP for that subject. Before using the same device for new patients, additional calibration is required to determine the formula for calculating intracranial pressure suitable for new patients.

The present invention does not exclude other ƒ(t_{ABP}) function forms other than formula (2).

The intracranial pressure monitoring device provided by the embodiment of the present invention specifically includes:

(1) Sensing probe: The probe is equipped with a digital-to-analog conversion module, a communication module and a rechargeable battery. The digital-to-analog conversion converts the electrical signal obtained by the sensing element into a digital signal, and the communication module transmits the digital signal to the host in real time. It is necessary to provide the power demand of the probe for a long enough time.

There are two types of connections between the communication module and the host:

1) Wired connection. Connect with the host through data cable and power cable. In this way, the power supply can be set in the host, reducing the weight and volume of the sensing probe, which is beneficial for the patient to wear. Compared with wireless transmission, the use of data lines can also transmit more excess signals in real time. The disadvantage of adopting this design scheme is that even if the main unit is small enough to be put into a coat pocket, the connecting wire between the main unit and the probe still has a certain influence on the life and work of the wearer. In addition, the data line must have good electromagnetic shielding, otherwise the electromagnetic interference generated by indoor power frequency signals and electromagnetic products (such as computers, microwave ovens) will greatly affect the signal quality acquired by the sensor.

2) Wireless connection. Use wireless transmission methods such as blue teeth to transmit signals between the host and the probe. The advantage of this scheme is that it is more convenient for patients to live and work, and when data is transmitted wirelessly, external electromagnetic interference is easily shielded. But the disadvantage is that the power supply (battery) must be set in the probe in the form of a scarf, neckband or necktie. The volume and weight of the rechargeable battery cannot be too large, but the power must be able to last for a long time. In addition, the distance between the host and the probe cannot exceed the maximum transmission distance adapted to the power of the Bluetooth module.

(2) Host: Receive the jugular pulsation signal detected by the sensor probe in real time through wired or wireless means. After the signal enters the processor for preliminary noise filtering and amplification, it is transmitted to the cloud data processing and storage center through Wi-Fi for further processing. Processing and calculation of intracranial pressure, the settlement results are sent back to the host computer via Wi-Fi for display.

(3) Cloud data processing and storage center: conduct in-depth analysis of the digital signals transmitted by the host computer or smart phone in real time, and calculate the statistical data of the patient's current intracranial pressure and intracranial pressure changes in the past period after removing interference signals, and then transmit back to the display on the host or smartphone.

The center will conduct an in-depth analysis of the digital signals transmitted by the host computer or smart phone in real time, remove various interference signals, and calculate the patient's current intracranial pressure and the changes in intracranial pressure in the past period according to the pre-input procedures and formulas. The statistical data is then transmitted back to the host or smartphone for display.

The center can store the data of multiple users, which is convenient for large medical institutions to call at any time, and facilitates the simultaneous monitoring and management of intracranial pressure of multiple patients. In this case, the host can be directly connected to the computer network, enter the hospital network system, and complete the processing, classification, mapping and storage of massive patient data.

(4) Product form

In order to prove the creativity and technical value of the technical solution of the present invention, this part is the application example of the claimed technical solution on specific products or related technologies.

### 1) Household Type

The user wears a light sensing element, and the host or smart phone receives and sends monitoring data to the cloud data center through Wi-Fi. The real-time monitoring data of intracranial pressure can be sent to the medical unit or the mobile phone of relatives at the user's discretion.

### 2) Medical Type

The product is used in medical units, and multiple sensing elements can be used to simultaneously collect jugular vein pulse signals of multiple patients. Then send them to the cloud data center or the mainframe of the medical unit through their respective bluetooth modules. The host is equipped with a multi-channel receiver, which processes the data of each patient at the same time and calculates the intracranial pressure in real time according to the installed software, and then displays it on the screen of the host. According to the intracranial pressure data of each patient, it can send the intracranial Warning of overpressure.

It should be noted that the embodiments of the present invention can be realized by hardware, software, or a combination of software and hardware. The hardware part can be implemented using dedicated logic; the software part can be stored in memory and executed by a suitable instruction execution system such as a microprocessor or specially designed hardware. Those of ordinary skill in the art will understand that the above-described devices and methods can be implemented using computer-executable instructions and/or contained in processor control code, for example, on a carrier medium such as a magnetic disk, CD or DVD-ROM, such as a read-only memory Such code is provided on a programmable memory (firmware) or on a data carrier such as an optical or electronic signal carrier. The device and its modules of the present invention can be realized by hardware circuits such as very big scale integrated circuits or gate arrays, semiconductors such as logic chips, transistors, etc., or programmable hardware devices such as field programmable gate arrays, programmable logic devices, etc., It can also be realized by software executed by various types of processors, or by a combination of the above hardware circuits and software such as firmware.

The embodiment of the present invention has achieved some positive effects in the process of research and development or use, and indeed has great advantages compared with the prior art. The following content is described in conjunction with data and charts of the test process.

In the case of the present invention, two piezoelectric transducers are used to measure the internal jugular vein pulse. Since the subjects hold their breath, the intracranial pressure will increase, and the harder they hold their breath, the greater the increase. Therefore, it is possible to compare the time difference between the corresponding pulsation feature points appearing on the two sensors in the state of not holding your breath and holding your breath.

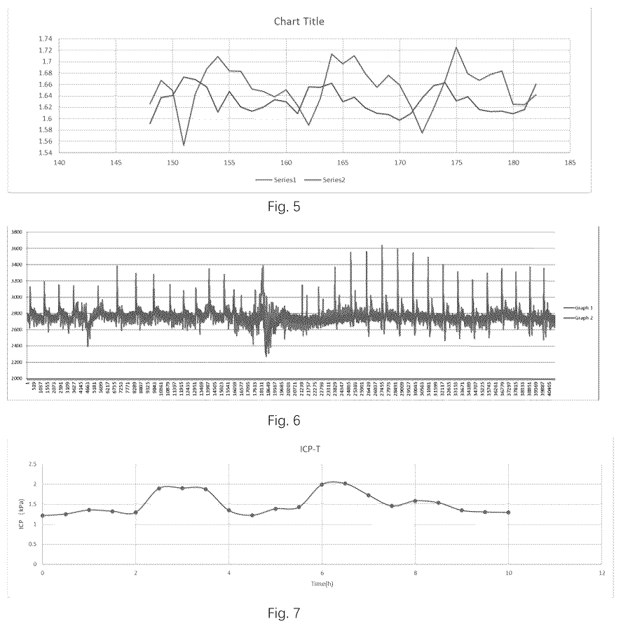

FIG. 6 is the amplitude-time curve of internal jugular vein pulse measured by volunteer A. The horizontal axis is the time axis. Because the sampling frequency is 1200/s, the time unit of the horizontal axis is 8.33*10-4 s. The vertical axis is the electrical signal strength axis. Since the signal strength does not enter the calculation formula of intracranial pressure, it can be used for it is amplified so that it is easier to distinguish the pulse cycle of the internal jugular vein and accurately locate the peak point of the wave. The blue curve in the figure is the amplitude-time curve of sensor Series1, and the red curve is the amplitude-time curve of sensor Series2. It can be seen that the setting position of sensor Series1 is closer to the heart than Series2, so its characteristic point (peak value of a wave) Appears sooner. Subtract the time t1 of Series1 from the time t2 of Series2, and the difference t21 should be greater than zero. The volunteers remained calm for a period of time after the start of the test, and held their breath at about 18280 points in the time axis, that is, 15 seconds after the start of the test. Hold breaths with rapid breath changes, then continue holding breaths until the end of the test.

The following table is the time and time difference (t21 value) of the a-wave peaks of the two sensors in each pulsation cycle obtained at each stage of this measurement. The first row of numbers in Table 1-Table 4 is the appearance of sensor Series2 The second row of numbers is the time when the sensor Series1 appears, and the third row of numbers is the t21 value.

TABLE 1
Calm phase

| | | | | | | | | | | | | | |
|---|---|---|---|---|---|---|---|---|---|---|---|---|---|
| 264 | 1354 | 2488 | 3630 | 5518 | 6999 | 8439 | 9823 | 11084 | 12132 | 13097 | 14123 | 15280 | 16621 |
| 252 | 1337 | 2475 | 3612 | 5506 | 6983 | 3427 | 9805 | 11076 | 12121 | 12076 | 14099 | 15265 | 16595 |
| 12 | 17 | 13 | 18 | 12 | 17 | 12 | 18 | 8 | 11 | 21 | 24 | 15 | 26 |

TABLE 2
Stages of forced breath holding

| | | | | | | | | | | |
|---|---|---|---|---|---|---|---|---|---|---|
| 18281 | 19946 | 21307 | 22574 | 23844 | 25073 | 26287 | 27491 | 28699 | 29884 | 31057 |
| 18281 | 19939 | 21307 | 22574 | 23837 | 25073 | 26286 | 27491 | 28697 | 29878 | 31051 |
| 0 | 7 | 0 | 0 | 7 | 0 | 1 | 0 | 2 | 6 | 6 |

TABLE 3
Rapid ventilation phase

| | | | |
|---|---|---|---|
| 32233 | 33376 | 34526 | 35652 |
| 32214 | 33351 | 34492 | 35628 |
| 19 | 25 | 34 | 24 |

TABLE 4
The stage of continuing to hold breath after ventilation

| | | | |
|---|---|---|---|
| 36761 | 37851 | 38947 | 40014 |
| 36754 | 37850 | 38936 | 40006 |
| 7 | 1 | 11 | 8 |

TABLE 5
The average value of t21 in each stage

| | | breath | | breath |
|---|---|---|---|---|
| Phase | calm | holding | ventilation | holding |
| Average t₂₁ (8.33*10⁻⁴ s) | 16.00 | 0.09 | 25.50 | 6.75 |

It can be seen from the above table that after holding breath, the time difference between the two sensors is greatly shortened, indicating that the pulse wave velocity of the internal jugular vein increases sharply, resulting in a sharp increase in intracranial pressure. During the brief ventilation period, due to the rapid relaxation of the internal jugular vein, the time difference increases rapidly, the pulse wave velocity drops suddenly, and the intracranial pressure also decreases rapidly. If you continue to hold your breath after exchanging breaths, the time difference is significantly reduced, but because the strength of holding your breath is not as strong as that of the first time, the increase in pulse wave velocity and intracranial pressure is not as good as the first time. In this case, because the sampling frequency is not high enough, the resolution is low, and the measurement range is narrow, so there will be a situation where t₂₁=0 in the breath-holding stage. This situation no longer occurs as the sampling frequency increases.

FIG. 7 is the time-domain diagram (ICP-T) of the change of intracranial pressure of volunteer B. Both transducers were sampled equally to measure internal jugular vein sampling. The sampling frequency reaches 2500/s. The horizontal axis in the figure is time (h), and the vertical axis is intracranial pressure (kPa). The volunteer stayed in bed for a long time. The calculation formula of the volunteer's intracranial pressure was determined according to the aforementioned calibration method, and then the intracranial pressure value was calculated every 30 minutes according to the mean value of the time difference between the a-wave peaks of the two sensors. It can be seen from the figure that the intracranial pressure of the volunteers is basically within the normal range (0.5-2.0 kPa). A significant increase in intracranial pressure occurred at about 2 hours because the volunteers changed to the lateral position. This is because although the craniospinal cavity is a closed cavity, it is not completely closed, and both the greater foramen magnum and the jugular foramen are affected by the external atmospheric pressure. The jugular vein may be compressed during recumbency, leading to a marked increase in intracranial pressure. After changing to the supine position for about 4 hours, the intracranial pressure decreased significantly. About 6 hours later, he changed to lie on his side again, and the intracranial pressure increased again. After being reminded by family members, the volunteers relaxed the pressure on the jugular vein, and the intracranial pressure returned to normal.

The above is only a specific embodiment of the present invention, but the protection scope of the present invention is not limited thereto. Anyone familiar with the technical field within the technical scope disclosed in the present invention, whoever is within the spirit and principles of the present invention Any modifications, equivalent replacements and improvements made within shall fall within the protection scope of the present invention.

What is claimed is:

1. An information processing method with intracranial pressure monitoring, is characterized in that, the information processing method with intracranial pressure monitoring comprises:
Step 1, Using at least two sensing probes, respectively fixed on the skin surface near the jugular vein, and the distance between the centers of each probe is greater than or equal to 2 mm;
Step 2: Sending the jugular pulsation signal measured by each probe to the host or smart phone via bluetooth or other wireless or wired methods at regular intervals;
Step 3. The host or smart phone analyzes the signals sent by each probe, finds out the corresponding feature points on the jugular vein pulsation map, and records the time when the same feature point appears on each probe for each pulsation;
Step 4, The programming software of the host computer calculates each beat and the time difference of the same feature point appearing on each probe, and calculates the mean value of the time difference of the same feature point appearing on each probe within the sampling time;
Step 5: Based on the average value of the time difference between the appearance of the same feature point on each probe, a mathematical formula for calculating the average intracranial pressure within the sampling time is established, and through the calibration process compared with other intracranial pressure measurement methods, the host programming software is used to determine the coefficients in the mathematical formula for calculating the average intracranial pressure.
Step 6, Based on the mathematical formula, the programming software of the host calculates the mean intracranial pressure in each sampling time in real time;
Step 7, By monitoring the change of the jugular venous pressure, the intracranial pressure change curve is obtained for reference.

2. The information processing method for monitoring intracranial pressure according to claim 1, wherein the sensing method adopted in the information processing method for monitoring intracranial pressure is piezoelectric sensing; the sensing probe adopts PVDF piezoelectric film;
The information processing method using intracranial pressure monitoring further includes acquiring a jugular vein pulsation map in a manner of array arrangement of sensing elements, and using a Venous imaging instrument to assist positioning of the sensor.

3. The information processing method for monitoring intracranial pressure according to claim 1, wherein the sensing probe includes piezoelectric, pressure sensing, Doppler ultrasonic sensing, photoelectric volume sensing and other Sensors used in pulse wave sensing technology.

4. The information processing method for intracranial pressure monitoring according to claim 1, wherein the jugular vein includes the internal jugular vein, the external jugular vein and other jugular veins;
The characteristic points include the highest point, the lowest point, the peak point of the V wave, or the time point when the pulse amplitude reaches a specified value in the jugular vein pulse diagram.

5. The information processing method for monitoring intracranial pressure according to claim 1, wherein the sampling method of the information processing method for monitoring intracranial pressure is:
Use the piezoelectric sensor to trace the electrical signal value generated by the jugular pulsation, use the electrical signal strength as the amplitude, and record the continuous jugular pulsation curve of the amplitude changing with time, and the sampling frequency of the electrical signal should not be lower than 500 Hz.

6. The information processing method for monitoring intracranial pressure according to claim 1, wherein the calculation process of the time difference is:
Put the jugular pulsation amplitude-time curves recorded by sensor probes A and B together, record the time t_{A} and t_{b} when the same characteristic point appears for each sensor probe in each beat, and calculate the time difference |tₐ−t_{b}|.
Next, continuously sample, record, and calculate the time difference (|tₐ−t_{b}|) for a given time period and use the arithmetic mean from the time difference values between all sensor probes as the average time difference t̅_̅{̅a̅b̅}̅ required for intracranial pressure calculation within the given period.
The intracranial pressure y is a function of t̅_̅{̅a̅b̅}̅, expressed as the following:
$$y = f ( \overline{{t}_{ab}} )$$
This function adopts quadratic form with the following formula:
$$y = {a ( \overline{{t}_{ab}} )}^{2} + b ( \overline{{t}_{a b}} ) + c$$
In this formula, a, b, and c are calibration coefficients calculated by comparing the intracranial pressure state below normal, normal, and above normal with the real intracranial pressure value.

7. An intracranial pressure monitoring device implementing the information processing method for monitoring intracranial pressure according to claim 1, characterized in that the intracranial pressure monitoring device comprises:
The host or smart phone is used to receive the jugular pulsation signal detected by the sensor probe in real time through wired or wireless means. After the signal enters the processor for preliminary noise filtering and amplification, it is transmitted to the cloud data processing and storage center through Wi-Fi. For further processing and calculation of intracranial pressure, the settlement result is sent back to the host computer for display via Wi-Fi;
Sensing probe, the sensing probe is in the form of a scarf, necktie or tie, equipped with a digital-to-analog conversion module, a communication module and a rechargeable battery, the digital-to-analog conversion converts the electrical signal obtained by the sensing element into a digital signal, and the communication module Real-time transmission of digital signals to the host;
The cloud data processing and storage center is used for in-depth analysis of the digital signals transmitted by the host computer or smart phone in real time, and calculates the statistical data of the patients current intracranial pressure and intracranial pressure changes in the past period after removing the interference signal, and then sends it back to the host or smartphone to display.

8. The intracranial pressure monitoring device according to claim 7, characterized in that, special software needs to be installed in the smart phone, and the software includes version A and version B, version A is suitable for Android mobile phone installation, and version B Applicable to the installation of Apple mobile phones;
The application process of the special software includes:
Establish an intracranial pressure detection interface on the smart phone, set personal information input, intracranial pressure calibration and display options on the interface; set up age, height, weight and other physiological parameter input columns in the personal information input options;
In the intracranial pressure display option, set up two display modes: the current intracranial pressure value and the curve of the intracranial pressure change trend in the past period;
At the same time, these data are also transferred to the hospital for reference when professionals treat and monitor the condition, and can also be used as a basis for emergency treatment.

9. An intracranial pressure monitoring device according to claim 8, characterized in that the sensor probe is pasted on the skin near the jugular vein of the patient with good biocompatibility adhesive, and the connection with the host includes two methods respectively:
(1) Wired connection, connected to the host through data cables and power cables, and setting the power supply in the host;
(2) Wireless connection, the Bluetooth wireless transmission method is used to transmit signals between the host and the probe, the power supply or battery is set in the sensing probe, the distance between the host and the probe cannot exceed the power adaptability of the Bluetooth module the maximum transmission distance.

10. An application for implementing the intracranial pressure monitoring device according to claim 7, characterized in that it is applied to family intracranial pressure or medical intracranial pressure monitoring;
The specific process of the family intracranial pressure monitoring is:
When wearing the sensor probe of the intracranial pressure monitoring equipment, the host or smart phone receives and sends the monitoring data to the cloud data processing and storage center through Wi-Fi, and can decide whether to send the real-time monitoring data of intracranial pressure to the medical unit or relative's mobile phone;
The specific process of the medical intracranial pressure monitoring is:
Multiple sensing elements are used to collect the jugular pulsation signals of multiple patients at the same time, and then send them to the cloud data processing and storage center or the large-scale host in the medical unit through their respective Bluetooth modules; the host is equipped with a multi-channel receiver to simultaneously process each According to the data of each patient, the intracranial pressure is calculated in real time according to the installed special software, and displayed on the host screen. According to the intracranial pressure data of each patient, a warning of intracranial pressure exceeding the limit is issued.
